(19) United States
(12) Patent Application Publication
(10) Pub. No.: US 2014/0066724 A1
(43) Pub. Date: Mar. 6, 2014

(54) System and Methods for Evaluating Vocal Function Using an Impedance-Based Inverse Filtering of Neck Surface Acceleration

(71) Applicants: Matias Zanartu, Lafayette, IN (US); Julio C. Ho, Chicago, IL (US); Daryush D. Mehta, Cambridge, MA (US); George R. Wodicka, West Lafayette, IN (US); Robert E. Hillman, Weston, MA (US)

(72) Inventors: Matias Zanartu, Lafayette, IN (US); Julio C. Ho, Chicago, IL (US); Daryush D. Mehta, Cambridge, MA (US); George R. Wodicka, West Lafayette, IN (US); Robert E. Hillman, Weston, MA (US)

(21) Appl. No.: 14/000,245

(22) PCT Filed: Feb. 20, 2012
(86) PCT No.: PCT/US2012/025817

§ 371 Date: Nov. 14, 2013

Related U.S. Application Data
(60) Provisional application No. 61/444,199, filed on Feb. 18, 2011.

(51) Int. Cl. A61B 5/00; A61B 5/107; A61B 5/087

(52) U.S. Cl. CPC A61B 5/725; A61B 5/087; A61B 5/7278; A61B 5/1075
USPC 600/301; 600/595

(57) ABSTRACT

A system and method to assess vocal function of a subject. The system includes an accelerometer configured to acquire surface acceleration data associated with vocal functionality of the subject and a computer system configured to analyze the surface acceleration data and to estimate glottal airflow wave-forms produced by the subject based on the surface acceleration data. The computer system performs the analysis and estimation by applying an inverse filter to the surface acceleration data based on a calibrated transmission line model and generates an indication of vocal functionality of the subject based on the estimated glottal airflow waveforms.

### CROSS-REFERENCE TO RELATED APPLICATIONS

This application is based on, claims the benefit of, and incorporates herein by reference U.S. Provisional Patent Application Ser. No. 61/444,199, filed on Feb. 18, 2011, entitled “Estimation of Glottal Aerodynamics Using an Impedance-Based Inverse Filtering of Neck Surface Acceleration.”

### STATEMENT REGARDING FEDERALLY SPONSORED RESEARCH

This invention was made with government support under R01 DC007640-01A2 awarded by the National Institutes of Health National Institute on Deafness and Other Communication Disorders. The government has certain rights in the invention.

### BACKGROUND OF THE INVENTION

The present application is directed to non-invasive estimation of vocal system operational parameters, such as glottal parameters used in the assessment of vocal function and, more particularly, a system and method for estimating glottal parameters using an impedance-based inverse filtering (IBIF) of neck surface acceleration.

Inverse filtering of speech sounds is used to estimate the source of excitation at the glottis (that is, the glottal source) and is based on source-filter theory principles to separate and remove the acoustic effects of the tracts from the source estimation. This technique is primarily performed for the vocal tract using recordings of oral airflow or radiated pressure, for example through closed phase inverse filtering (CPIF). Oral airflow or pressure recordings require use of a circumferentially-vented mask, and thus, are only suitable for use in clinical settings. However, commonly-occurring voice disorders are difficult to assess in the clinic and could potentially be much better characterized by long-term ambulatory monitoring of vocal function as subjects engage in their typical daily activities.

Accordingly, other types of inverse filtering techniques have been implemented, for example, that rely on acceleration measured on the skin overlying the suprasternal notch to obtain estimates of glottal parameters. However, this technique, which relies on so-called subglottal inverse filtering, requires a different approach than what is used for oral airflow or pressure measurements, making standard vocal tract-based methods inapplicable. To date, these attempts have been limited by the partial understanding of the underlying physical phenomena and necessary parameters, and thus, the factors that could distort the estimates.

Therefore, it would be desirable to provide a system and method for accurate estimation of various operation parameters for assessment of vocal function.

### SUMMARY OF THE INVENTION

The present invention overcomes the aforementioned drawbacks by providing a model-based scheme for an accurate, non-invasive estimation of clinical parameters used in the ambulatory assessment of vocal function. The model-based scheme allows for subject-specific calibration protocols and accounts for a variety of variations in data acquisition, data analysis, and ultimate reporting of vocal function. The approach, referred to as impedance-based inverse filtering(IBIF), takes as input the signal from a light-weight accelerometer placed on the skin over the extrathoracic trachea and yields estimates of glottal airflow and its derivative. IBIF is based on impedance representations obtained via mechano-acoustic analogies and a physiologically-based transmission line model. The transmission line model represents the subglottal system divided between portions below and above the accelerometer location and includes a neck skin model based on lumped representations. A subject-specific calibration protocol is used to account for individual adjustments of subglottal impedance parameters and mechanical properties of the skin. No glottal coupling is required as the subglottal model transfers all source-filter interaction effects into the glottal source.

In accordance with one aspect of the invention, a method for evaluating vocal function of a subject includes collecting surface acceleration data from an accelerometer coupled to a neck of the subject and obtaining at least one other physiological indication signal from the subject. The method also includes applying an inverse filter to the neck surface acceleration data based on a basis transmission line model to obtain an estimated glottal airflow waveform, comparing at least one portion of the estimated glottal airflow waveform to the at least one other physiological signal, and adjusting at least one parameter of the basis transmission line model based on the comparison step to yield a calibrated transmission line model. The method further includes reapplying the inverse filter to the surface acceleration data based on the calibrated transmission line model to obtain a new estimated glottal airflow waveform, repeating at least a portion of the previous steps and analyzing at least one portion of the new estimated glottal airflow waveform against at least a portion of the estimated glottal airflow waveform, and generating an indication of vocal function of the subject based on the analysis.

In accordance with another aspect of the invention, a system to assess vocal function of a subject is disclosed. The system includes an accelerometer configured to acquire surface acceleration data associated with vocal functionality of the subject and a computer system configured to analyze the surface acceleration data and to estimate glottal airflow waveforms produced by the subject based on the surface acceleration data. The computer system performs the analysis and estimation by applying an inverse filter to the surface acceleration data based on a basis transmission line model to obtain a first glottal waveform output, comparing at least one portion of the first glottal waveform output to at least one other physiological signal of the subject, and adjusting at least one parameter in the basis transmission line model based on the comparison step to obtain a calibrated transmission line model. The computer system then reapplies the inverse filter to the neck surface acceleration data based on the calibrated transmission line model to obtain the estimated glottal airflow waveforms and generates an indication of vocal functionality of the subject based on the estimated glottal airflow waveforms.

These and other features and advantages of the present invention will become apparent upon reading the following detailed description when taken in conjunction with the drawings.

### BRIEF DESCRIPTION OF THE DRAWINGS

FIG. 1a is a schematic drawing of an acoustic transmission-line model representing impedances of the subglottal tract;

FIG. 1b is a schematic drawing of an equivalent two-port symmetric representation of the acoustic transmission line model in FIG. 1a;

FIG. 2 is a flow chart of steps performed in accordance with one implementation of the present invention;

FIG. 3 is an illustration of the subglottal system;

FIG. 4 is a schematic of a dipole model representation of the subglottal system of FIG. 3 using two ideal airflow sources;

FIGS. 5a and 5b are graphs of experimental results illustrating estimates of glottal airflow (Uₛᵤₚᵣₐ) and its derivative (dUₛᵤₚᵣₐ), respectively, obtained from measurements of neck surface acceleration and impedance-based inverse filtering (ACC) and from measurements of oral airflow and closed-phase inverse filtering (CPIF) for sustained vowel /a/ in the chest register;

FIGS. 5c and 5d are graphs of experimental results illustrating estimates of glottal airflow (Uₛᵤₚᵣₐ) and its derivative (dUₛᵤₚᵣₐ), respectively, obtained from measurements of neck surface acceleration and impedance-based inverse filtering (ACC) and from measurements of oral airflow and closed-phase inverse filtering (CPIF) for sustained vowel N in the chest register;

FIGS. 6a and 6b are graphs of experimental results illustrating estimates of glottal airflow (Uₛᵤₚᵣₐ) and its derivative (dUₛᵤₚᵣₐ), respectively, obtained from measurements of neck surface acceleration and impedance-based inverse filtering (ACC) and from measurements of oral airflow and closed-phase inverse filtering (CPIF) for sustained vowel /a/ in the falsetto register; and

FIGS. 6c and 6d are graphs of experimental results illustrating estimates of glottal airflow (Uₛᵤₚᵣₐ) its derivative (dUₛᵤₚᵣₐ), respectively, obtained from measurements of neck surface acceleration and impedance-based inverse filtering (ACC) and from measurements of oral airflow and closed-phase inverse filtering (CPIF) for sustained vowel N in the falsetto register.

### DETAILED DESCRIPTION OF THE INVENTION

The present invention provides a model-based inverse filtering scheme that allows for an enhanced estimation of glottal airflow from acceleration measurements of the skin overlying the sternal notch. The scheme, referred to as impedance-based inverse filtering (IBIF), is based on mechano-acoustic analogies, transmission line principles, and physiological descriptions. The scheme can be used to evaluate the effects of source-filter interactions due to incomplete glottal closure on subglottal and supraglottal inverse filtering, can help determine whether glottal coupling is needed to retrieve the “true” glottal airflow, and/or can be applied to the estimation of the glottal source from measurements of neck surface acceleration. The scheme can be used to evaluate the effects of source-filter interactions due to incomplete glottal closure on subglottal and supraglottal inverse filtering, can help determine whether glottal coupling is needed to retrieve the “true” glottal airflow, and/or can be applied to the estimation of the glottal source from measurements of neck surface acceleration

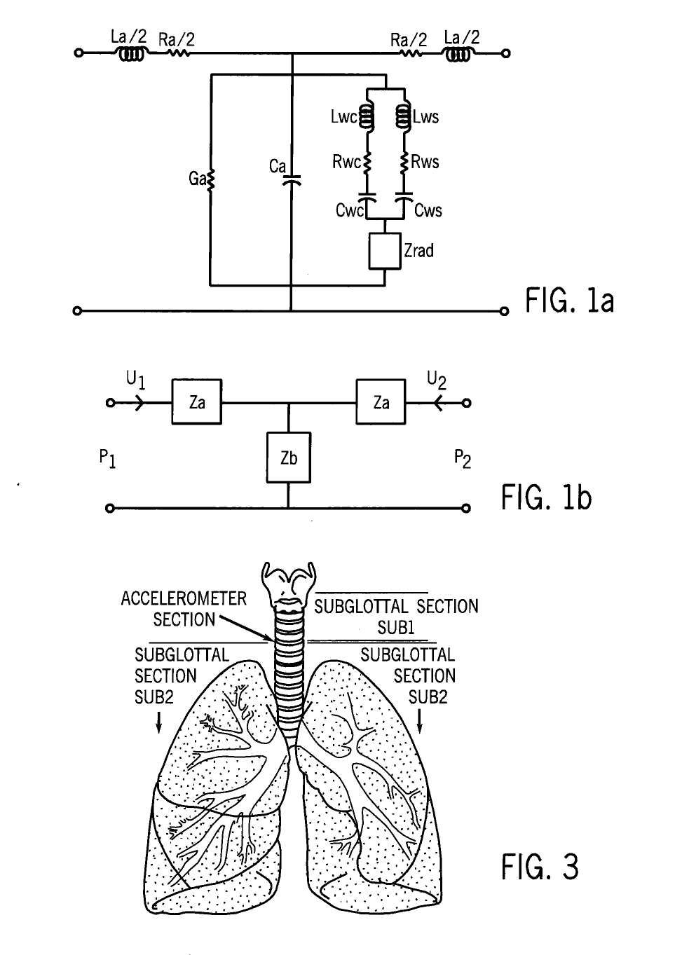

The scheme considers a model, or module, of system impedances for the subglottal tract, separate from the supraglottal tract and the glottis, which can be estimated from observed signals to obtain subject-specific values. In order to estimate the subglottal tract impedances, a model of acoustic transmission can be applied, as shown in FIG. 1a. The acoustic transmission line model illustrated in FIG. 1a incorporates air inertance Lₐ, air viscous resistance Rₐ, heat conduction resistance Gₐ, and air compliance Cₐ, which are considered acoustical representations for losses, elasticity, and inertia. In addition, FIG. 1a incorporates impedances based on yielding walls of the subglottal system, including cartilage components of inertance, resistance, and compliance (L_{wc}, R_{wc}, C_{wc}, respectively) and soft tissue components of inertance, resistance, and compliance (L_{wc}, R_{wc}, C_{wc}, respectively). Also, a radiation impedance Z_{rad} is used to account for skin neck properties and loading of the accelerometer (for example, a surface bioacoustical sensor) used for acquiring neck skin acceleration data.

FIG. 1b illustrates an equivalent two-port symmetric representation of the model of FIG. 1a. The acoustic transmission line model of FIG. 1b is based on a series of concatenated T-equivalent segments of lumped acoustic elements that relate acoustic pressure (P(ω)) to volume velocity (U(ω)) and can be used to compute transmission line parameters. For example, in the illustrated representation, a cascade connection is used to account for the acoustic transmission matrix associated with each section represented by the two-port T-network. This approach provides relations for both P(ω) and U(ω), so that a flow-flow transfer (H(ω)) or driving-point input impedance (Zᵢₙ(ω)) function can be computed for the subglottal tract. As shown in FIG. 1b, the equivalent impedance of the shunt terms in FIG. 1a is denoted as Z_{b}, and that of the series term on each side in FIG. 1a is denoted as Zₐ. With reference to the circuit of FIG. 1b, the symmetric transmission matrix that relates two neighboring T-sections has the following structure (also known as an ABCD network):

$$[ \begin{matrix}{P}_{1} ( ω ) \\ {U}_{1} ( ω )\end{matrix} ] = [ \begin{matrix}A & B \\ C & D\end{matrix} ] [ \begin{matrix}{P}_{2} ( ω ) \\ - {U}_{2} ( ω )\end{matrix} ] , \qquad ( 1 )$$

where both flows are considered to enter the T-section, so that

A=(Z₁+Z_{b})Z_{b}⁻¹ (2);

B=(Zₐ+2Z_{b})Z₁Z_{b}⁻¹ (3);

C=Z_{b}⁻¹ (4);

D 32 A. (5);

Thus, the flow transfer function H(ω)U₂/U₁₁ is given by:

$$H ( ω ) = \frac{1}{{cz}_{2} ( ω ) + D} ; \qquad ( 6 )$$

and the driving point impedance from the first section or input impedance Z₁(ω) by:

$${Z}_{1} ( ω ) = \frac{{AZ}_{2} ( ω ) + B}{{CZ}_{2} ( ω ) + D} ; \qquad ( 7 )$$

where Z₂(ω) acts as the effective load impedance for the two-port network. As either cascade or branching configurations are commonly encountered in the subglottal tract, the network is solved by carrying the equivalent driving-point impedance of previous tracts, starting with a radiation or terminal impedance and ending at the glottis. This allows for the inclusion of subglottal branching in the subglottal system without increasing the complexity of the overall approach. The transmission line model derived above can yield the driving point impedance as well as a transfer function for any desired location within the tract. These terms only depend on the tract configuration and its inherent physical properties.

In some implementations of the invention, as described above, an estimation of the glottal airflow based on non-invasive measurements can be obtained through neck surface acceleration measured through the extrathoracic trachea at the level of the suprasternal notch. To execute this estimation, the subglottal tract transmission line model can receive as input an accelerometer signal and can output an airflow waveform just below the glottis, which can be denoted as \dot{U}skin and U_{sub}, respectively. The frequency domain transfer function between these signals, Tₛₖᵢₙ=\dot{U}ₛₖᵢₙ/U_{sub}, can be obtained through the subglottal tract module and then inverted to estimate the glottal airflow from neck surface acceleration.

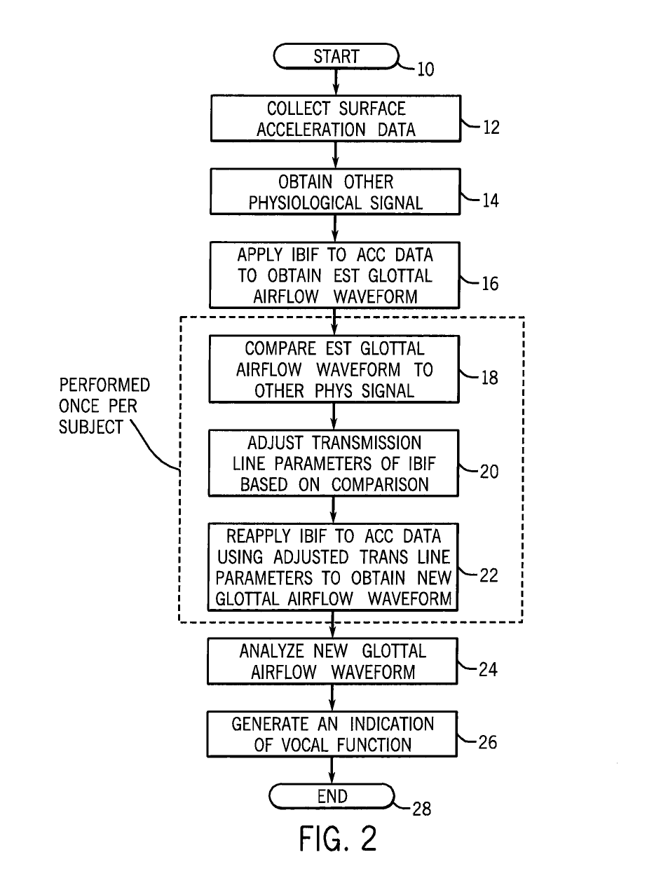

FIG. 2 illustrates an example procedure for estimating glottal airflow according to the present invention. The steps are first described generally and then in more detail in the following paragraphs. After starting the procedure (process block 10), surface acceleration data is collected through the accelerometer positioned over the suprasternal notch (process block 12). At least one other physiological signal can then be obtained or collected for calibration purposes (process block 14). As will be described, this other physiological signal may include a first resonance frequency obtained from the surface acceleration data, an oral airflow waveform, and/or any of a wide variety of other parameters further detailed below. The IBIF is applied to the surface acceleration data based on a basis subglottal transmission line model to obtain an estimated glottal airflow waveform (process block 16). A portion of the estimated glottal airflow waveform is compared to the other physiological signal (process block 18) and then parameters of the basis transmission line model are adjusted based on the comparison to obtain a calibrated transmission line model with subject-specific parameters (process block 20). This adjustment can be performed with any multimodal optimization scheme (for example, Particle Swarm Optimization). For all subsequent uses, the IBIF is then reapplied to the surface acceleration data based on the calibrated transmission line model to obtain a new, calibrated glottal airflow waveform (process block 22). The new glottal airflow waveform and/or its derivative can then be analyzed (process block 24) and an indication of vocal function can be generated (process block 26). The procedure is then completed (process block 28). In some implementations of the invention, the above steps of the process illustrated in FIG. 2 can be executed by a computer system. In addition, in some implementations of the invention, calibration (in particular, process blocks 18-22) can be performed once per subject. In subsequent procedures after calibration has been performed, the IBIF applied in process block 16 can be based on the calibrated transmission line model, process blocks 18-22 can be omitted, and the glottal airflow waveform obtained in process block 16 can be analyzed in process block 24.

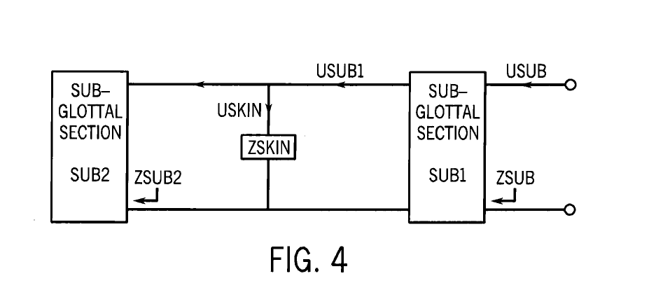

With reference to process block 12 above, FIG. 3 illustrates an anatomical representation of the subglottal system. As shown in FIG. 3, the accelerometer can be placed on the skin surface overlying the suprasternal notch at approximately 5 cm below the glottis. The subglottal tract can be decomposed into two subglottal sections, Sub₁ and Sub₂, that represent the portion of the extrathoracic trachea above and below the accelerometer, respectively. With reference to the transmission line models of process blocks 16 and 22, FIG. 4 illustrates a corresponding T-network of the two subglottal subsections. The section where the accelerometer is positioned is also represented in the T-network between the two subglottal sections (that is, at the location of Zₛₖᵢₙ), as shown in FIG. 4. The corresponding tract subsections can include driving point impedances Z_{sub1} and Z_{sub2}. In light of the model shown in FIG. 4, the volume velocity U ₛₖᵢₙ flowing through Zₛₖᵢₙ can be expressed as:

$${U}_{skin} ( ω ) = {U}_{sub 1} \frac{{z}_{sub 2}}{{z}_{sub 2} + {z}_{skin}} , \qquad ( 8 )$$

where Zₛₖᵢₙ is determined as the mechanical impedance of the skin Zₘ (based on skin resistance Rₘ, skin mass Mₘ, and skin stiffness Kₘ) in series with the radiation impedance Z_{rad} due to the accelerometer loading. Thus,

$$\begin{matrix}{z}_{skin} ( ω ) = {z}_{m} + {z}_{rad} , & ( 9 ) \\ {z}_{m} ( ω ) = {R}_{m} + j ( ω {M}_{m} = \frac{{κ}_{m}}{ω} ) , and & ( 10 ) \\ {z}_{rad} ( ω ) = \frac{jω {M}_{acc}}{{A}_{acc}} . & ( 11 )\end{matrix}$$

The skin volume velocity can be differentiated to obtain the neck surface acceleration signal \dot{U}ₛₖᵢₙ. Therefore, the transfer function between the subglottal volume velocity and the acceleration signal, referred to as Tₛₖᵢₙ, can be expressed as:

$${T}_{skin} ( ω ) = \frac{{\dot{U}}_{skin}}{{U}_{sub}} = \frac{{H}_{sub 1} \cdot {z}_{sub 2} \cdot {H}_{d}}{{z}_{sub 2} + {z}_{skin}} , \qquad ( 12 )$$

where H_{sub1}=U_{sub1/U}_{sub} is the transfer function of the subglottal section Sub₁ from the glottis to the acceleration location, and H_{d}=jω is the ideal derivative filter. In some implementations, it can be convenient to directly estimate the airflow entering the vocal tract Uₛᵤₚᵣₐ, which is related to the subglottal airflow using Uₛᵤₚᵣₐ=−U_{sub}. Thus, estimation of the airflow entering the vocal tract requires inverting the subglottal transfer function (that is, Uₛᵤₚᵣₐ=−\dot{U}ₛₖᵢₙ/Tₛₖᵢₙ). To avoid artifacts introduced by the low-frequency content of the subglottal impedance (|Z_{sub}(0)|→0), the gain of the transfer function Tₛₖᵢₙ can be set to be always larger or equal than one. The inverse filtering process can be performed in the frequency domain using the fast Fourier transform (FFT) and its inverse. Reconstruction with real output can be achieved by setting the FFT resolution to be at least the number of samples in \dot{U}ₛₖᵢₙ and forcing Tₛₖᵢₙ to be symmetric. This approach can also be implemented using periodic windowing and overlap-add reconstruction.

A default transmission line parameter set can be utilized in the basis transmission line model of process block 16 (for example, based on previously determined values). For example, the equations used to determine the parameters L_{α}, R_{α}, G_{α}, and C_{α} are shown below in Table I and are considered lumped parameters for a lossy rigid-walled transmission line segment.

TABLE I
LUMPED PARAMETERS FOR A LOSSY RIGID-WALLED
TRANSMISSION LINE SEGMENT

| Parameter | Value | Units |
|---|---|---|
| Resistance | ${R}_{a} = \frac{2 l}{π {r}^{3}} \sqrt{\frac{{ωρ}_{0} η}{2}}$ | $\frac{dyne \cdot s}{{cm}^{5}}$ |
| Inertance | ${L}_{a} = \frac{{ρ}_{0} l}{A}$ | $\frac{dyne \cdot {s}^{2}}{{cm}^{5}}$ |
| Compliance | ${C}_{a} = \frac{Al}{{ρ}_{0} {c}^{2}}$ | $\frac{{cm}^{5}}{dyne}$ |
| Conductance | ${G}_{a} = 2 π rl \frac{v - 1}{{ρ}_{0} {c}^{2}} \sqrt{\frac{κω}{2 {c}_{p} {ρ}_{0}}}$ | $\frac{{cm}^{5}}{dyne \cdot {s}^{5}}$ |

Variables in Table I are defined as follows: r=tube radius [cm]; l=segment length [cm]; ω=radian frequency; ρₒ=density of median [g/cm³]; η=shear viscosity [dyne s/cm²]; A=cross-sectional area [cm²]; c=speed of sound [cm/s]; ν=ratio of specific heats; κ=heat conduction coefficient [cal/cm-s−° C]; and cₚ=specific heat at constant pressure [cal/g−° C]. Physical properties of air are defined in Table II below:

TABLE II
PHYSICAL PROPERTIES OF AIR

| Property | Air | |
|---|---|---|
| ρ_{ο} (g/cm³) | 1.14 · | 10⁻³ (moist air, 37° C.) |
| η (dyne s/cm²) | 1.86 · | 10⁻⁴ (20° C., 1 atm) |
| υ = cₚ/c_{ν} | 1.4 | |
| κ (cal/cm-s-° C.) | 0.064 · | 10⁻³ (37° C.) |
| cₚ (cal/g-° C.) | 0.24 (0° C., 1 atm) | |
| c (cm/s) | 3.54 · | 10⁴ (moist air, 37° C.) |

The equations used to estimate the cartilage component parameters L_{wc}, R_{wc}, C_{wc} and the soft tissue component parameters L_{ws}, R_{ws}, C_{ws} are shown below in Table Ill and are considered lumped parameters for a nonrigid-walled transmission line segment of length, l.

TABLE III
NONRIGID WALL, LUMPED PARAMETERS FOR A SEGMENT OF
LENGTH l

| Parameter | Value | Units |
|---|---|---|
| Resistance | ${R}_{wx} ( ω ) = \frac{{η}_{wx} ( ω ) h}{2 π {r}^{3} l}$ | $\frac{dyne \cdot s}{{cm}^{5}}$ |
| Inertance | ${L}_{wx} ( ω ) = \frac{{ρ}_{wx} h}{2 π rl}$ | $\frac{dyne \cdot {s}^{2}}{{cm}^{5}}$ |
| Compliance | ${C}_{wx} ( ω ) = \frac{2 π {r}^{3} l}{{E}_{wx} ( ω ) h}$ | $\frac{{cm}^{5}}{dyne}$ |

Parameters in Table III are used for both soft tissue and cartilage, where the “x” value in the subscript is either an “s” (soft tissue) or a “c” (cartilage) for any given definition. Variables in Table III are defined as follows: r =tube radius [cm]; l=segment length [cm]; ω=radian frequency; and h=wall thickness [cm]. Tissue properties are: η_{wx}=shear viscosity [dyne s/cm²]; ρ_{wx}=density [g/cm³]; and E_{wx}=elasticity [dyne/cm²]. The tissue-specific values for η_{wx}, ρ_{wx}, and E_{wx} are defined in Table IV below:

TABLE IV
DEFAULT WALL PARAMETER VALES FOR
RESPIRATORY TRACT

| Parameter | Default Value | |
|---|---|---|
| Thickness (h) | 0.5 | cm |
| Soft Tissue Density (ρ_{ws}) | 1.06 | g/cm³ |
| Soft Tissue Viscosity (η_{ws}) | 1.6 · 10³ | dyne s/cm² |
| Soft Tissue Elasticity (Ε_{ws}) | 0.392 · 10⁶ | dyne/cm² |
| Cartilage Density (ρ_{wc}) | 1.14 | g/cm³ |
| Cartilage Viscosity (η_{wc}) | 180.0 · 10³ | dyne s/cm² |
| Cartilage Elasticity (Ε_{wc}) | 44.0 · 10⁶ | dyne/cm² |

In one implementation, the acoustic transmission line model of a symmetric branching subglottal representation from previous studies may be used as the basis subglottal transmission line model in process block 16. In particular, symmetric anatomical descriptions for an average male are used, since it yields overall values reported experimentally. One example of these values are presented in Table V below. In addition, default mechanical properties for the neck skin (for example, from previous studies) can be used. The default mechanical properties can include per unit area values of Rₘ=2320 grams/second, Mₘ=2.4 grams, Kₘ=491,000 dyne/centimeter. Mechanical properties for the accelerometer loading can be based on the light-weight accelerometer Knowles BU-7135, with a mass per unit area of M_{acc}/A_{acc}=0.26 grams. Also, the placement of the accelerometer over the suprasternal notch is initially assumed to be located at five centimeters below the glottis.

TABLE V
AIRWAY SEGMENT PARAMETERS FOR THE SUBGLOTTAL
TRACT STARTING AT THE TRACHEA (DEPTH 0)

| | Tube Length, l | Tube Radius, r | Wall | Fraction of |
|---|---|---|---|---|
| Depth | [cm] | [cm] | thickness, h | cartilage, c_{trac} |
| 0 | 10.0 | 0.80 | 0.3724 | 0.67 |
| 1 | 5.0 | 0.6 | 0.1735 | 0.5000 |
| 2 | 2.2 | 0.55 | 0.1348 | 0.5000 |
| 3 | 1.1 | 0.40 | 0.0528 | 0.3300 |
| 4 | 1.05 | 0.365 | 0.0409 | 0.2500 |
| 5 | 1.13 | 0.295 | 0.0182 | 0.2000 |
| 6 | 1.13 | 0.295 | 0.0182 | 0.0922 |
| 7 | 0.97 | 0.270 | 0.0168 | 0.0848 |
| 8 | 1.08 | 0.215 | 0.0137 | 0.0669 |
| 9 | 0.950 | 0.175 | 0.0114 | 0.0525 |
| 10 | 0.860 | 0.175 | 0.0114 | 0.0525 |

The basis subglottal transmission line model can be calibrated in process blocks 18 and 20 to match subject-specific parameters and obtain a calibrated transmission line model for use in process block 22 using one or both of the following approaches: a resonance matching approach and a waveform matching approach. The resonance matching approach is achieved by comparing, at process block 18, a first resonance of the estimated airflow waveform to a first subglottal resonance measured from the accelerometer signal (that is, the other physiological signal obtained in process block 14) and adjusting the model output to match the first subglottal resonance measured at process block 20. In particular, the segment length of the trachea, considered to be the primary anatomical difference between subjects in the lower airways, is modified to adjust the model parameters at process block 20 and produce the observed resonance. The first accelerometer resonance is obtained via the covariance method of linear prediction during the closed phase of the cycle. Even though it is known that this method fails to describe the zeros from the subglottal impedance, preliminary testing with human data and synthetic speech showed that it was sufficiently accurate and stable to estimate the frequency of the first subglottal resonance.

The waveform matching approach uses a minimum mean squared error scheme to account for variation of the tissue properties among subjects and/or other parameters, such as segment length of the trachea and accelerometer location. In the waveform matching approach, the parameters are adjusted to match oral airflow waveforms translated to glottis. For example, oral airflow waveform signals can be measured from a circumferentially vented mask (that is, the other physiological signal obtain at process block 14). The measured oral airflow waveform and the estimated glottal waveform output can be aligned, at process block 18, and the parameters are selected to minimize the root mean squared error (RMSE) at process block 20. Other potential subject-specific differences, such as tracheal diameter and losses in the subglottal system, can be compensated with this waveform matching approach and added as part of the mechanical properties of the skin. In some implementations, parameter limits can be applied to avoid model overfitting and to keep the model physiologically meaningful. For example, the accelerometer location can be constrained to about two centimeters above or below the initial location at five centimeters below the glottis. In addition, the tracheal length can be constrained so that it cannot be varied more than 50%, and the skin properties (inertance, resistance, and compliance), can be constrained so that they cannot vary more than ten times their default values.

After applying one or both of the calibration approaches, the calibrated transmission line model can then be used to apply the IBIF to the surface acceleration data and obtain a new glottal waveform estimate at process block 22. The new glottal waveform estimate and/or its derivative can be analyzed at process block 24, as further described below, and an indication of vocal function can be generated at process block 26, such as an indication whether vocal hyperfunction is present.

The following paragraphs describe an experiment used to evaluate the IBIF scheme of the present invention. The experiment described below is an evaluation of actual recordings of sustained vowels. This experimental approach provides different quantifiable glottal configurations during normal phonation of sustained vowels /a/ and /i/. Selected measures of glottal behavior from the actual recordings can be used to explore the ability of the IBIF scheme to correctly estimate the main characteristics of the glottal source. The selected measures of glottal behavior include the difference between the first two harmonics (H2−H1), harmonic richness factor (HRF), amplitude of the unsteady airflow (AC flow), and maximum flow declination rate (MFDR). In clinical use, these selected measures may be output as indications of vocal function (for example, at process block 26 in the process of FIG. 2). Errors determined in experimental results described below are presented with respect to a given reference signal, where the absolute difference and its ratio with respect to the reference are employed.

The goal of the actual speech recording evaluation was to obtain estimates of the complete system behavior through simultaneous recordings of vibration, glottal behavior, flow aerodynamics, and acoustic pressures. Thus, the experimental setup considered synchronous measurements of skin surface acceleration (ACC), oral volume velocity (OVV), electroglottography (EGG), and radiated acoustic pressure (MIC).

The OW was obtained through a circumferentially-vented (CV) mask (model MA-IL, Glottal Enterprises) that was modified to allow for adequate placement of the flexible endoscope with sufficient mobility while maintaining a proper seal. Calibration of the OW signal was performed by airflow calibration unit (Model MCU-4, Glottal Enterprises) after each recording session.

The ACC signal was obtained using a light-weight accelerometer (model BU-7135; Knowles) attached to the skin overlying the suprasternal notch (five centimeters below the glottis) using double sided tape (No. 2181, 3M). The accelerometer at this location provides good tissue-borne sensitivity and is essentially unaffected by normal background noise. The accelerometer was calibrated using a laser vibrometer.

The MIC signal was recorded using a head-mounted, high-quality condenser microphone (model MKE104, Sennheiser electronic GmbH & Co. KG). Calibration of the MIC signal was performed after each recording session by comparing side-by-side recordings of a stable wideband reference tone generator (COOPER-RAND, Luminaud, Inc.) with the MIC signal and a Class-2 sound level meter (Model NL-20, RION Co.) set to linear “C” weighting and “Fast” response time. No calibration of the EGG was undertaken in this experiment.

The protocol for this experiment required a subject uttering two sustained vowels (/a/ and /i/) and three different glottal conditions (breathy, chest, falsetto). Two subjects, a male with no vocal training and a female with vocal training, completed the required calibrated, synchronous recording sessions. These subjects had no history of vocal pathologies and were in the 28-34 age range. All recordings were obtained in an acoustically treated room at the Laryngeal Surgery & Voice Rehabilitation Center at the Massachusetts General Hospital.

As described above, the focus of the actual voice recording evaluation was to obtain estimates of glottal airflow parameters from the neck surface acceleration signal in real speech recordings. According to the present invention, the ability to obtain estimates of airflow that is entering the vocal tract does not depend on the glottal configuration or glottal coupling. Therefore, only the subglottal module is needed for the estimation of the desired glottal airflow (Uₛᵤₚᵣₐ) via measurement of neck surface acceleration, without requiring additional coupling of a subglottal or glottal module. This can hold true even under incomplete glottal closure scenarios. The present invention utilizes this discovery to create a modeling mechanism that is not encumbered by unnecessary parameters and, thereby, is readily utilized to evaluate vocal performance, including user-specific calibration, in a manner that is highly effective and efficient.

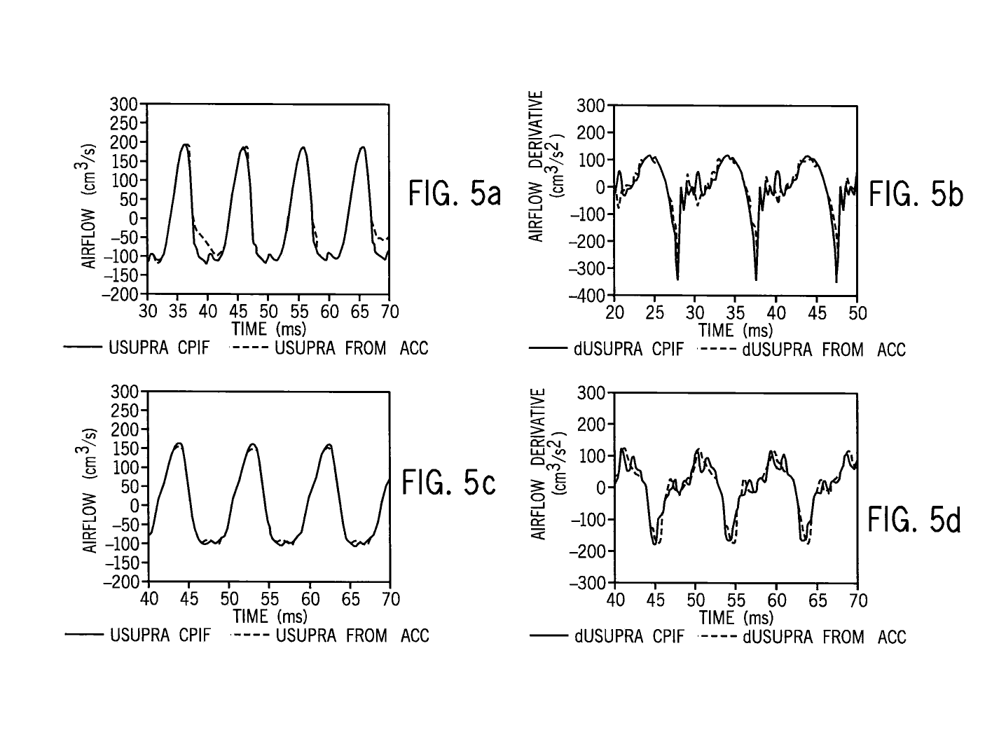

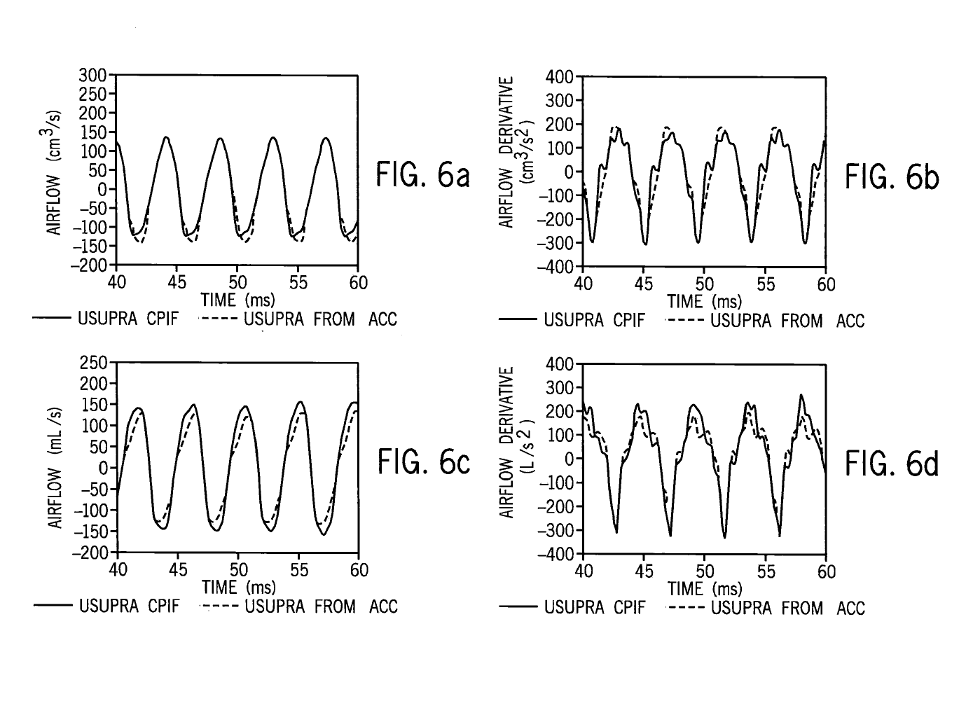

Estimates of glottal airflow (Uₛᵤₚᵣₐ) and its derivative (dUₛᵤₚᵣₐ) were obtained from the ACC signal and IBIF and contrasted with those inverse filtered from the vocal tract using the current criterion standard of CV mask airflow measurements and CPIF. The raw waveforms for these cases are presented for vowels /a/ and /i/ in chest register in FIGS. 5a-5d and falsetto register in FIGS. 6a-6d. It is noted that the ACC estimates in FIGS. 5a-5d and 6a-6d have no DC component. The degree of incomplete glottal closure, vibratory mode, and fundamental frequency change between these two registers. It is noted from these figures that the ACC-based waveforms were very similar to the OVV-based ones, with an error that appeared to vary between the glottal conditions and vowels. It was also observed that the closest waveform match was obtained during the open phase portion of the cycle for all cases.

A quantitative analysis of the measures extracted for all cases and subjects under evaluations (that is, 14 cases with at least 10 observations on each case) is presented in Table V. It was observed that for the normal chest voice in vowel /a/, the measures were within the expected range for male and female cases from previous studies. The vowel /i/ has not been previously studied and thus has no reference for comparisons.

TABLE V
RAW DATA FROM CPIF (1) AND ACC (2) MEASURES OF GLOTTAL BEHAVIOR.
MEASURES WERE OBTAINED OVER AT LEAST 10 CYCLES FOR EACH CASE.

| | Measure | | | | | | | | | | | | | |
|---|---|---|---|---|---|---|---|---|---|---|---|---|---|---|
| | Female subject | | | | | | Male subject | | | | | | | |
| | Chest | | Breathy | | Falsetto | | Chest | | Breathy | | Falsetto | | Chest | |
| | /a/ | /i/ | /a/ | /i/ | /a/ | /i/ | /a/ | /i/ | /a/ | /i/ | /a/ | /i/ | /a/ | /i/ |
| fo | 225 | 229 | 229 | 237 | 488 | 481 | 117 | 115 | 120 | 117 | 227 | 225 | 103 | 107 |
| RMSE Uₛᵤₚᵣₐ | 24 | 29 | 18 | 9 | 14 | 19 | 24 | 15 | 11 | 10 | 15 | 33 | 26 | 13 |
| RMSE dUₛᵤₚᵣₐ | 81 | 162 | 22 | 29 | 72 | 110 | 48 | 23 | 21 | 18 | 50 | 58 | 47 | 27 |
| RMSE Uₘ | 62 | 68 | 18 | 21 | 107 | 71 | 27 | 15 | 17 | 15 | 52 | 16 | 268 | 14 |
| AC flow 1 | 286 | 320 | 202 | 122 | 123 | 140 | 230 | 147 | 150 | 128 | 270 | 302 | 312 | 269 |
| AC flow 2 | 297 | 371 | 204 | 119 | 127 | 144 | 185 | 150 | 133 | 136 | 282 | 246 | 344 | 263 |
| MFDR 1 | 467 | 558 | 177 | 142 | 304 | 406 | 214 | 102 | 85 | 80 | 380 | 340 | 351 | 175 |
| MFDR 2 | 428 | 617 | 187 | 140 | 342 | 439 | 192 | 129 | 72 | 76 | 328 | 337 | 336 | 196 |
| H2-H1 1 | −15 | −9 | −26 | −10 | −9 | −5 | −10 | −12 | −23 | −17 | −16 | −21 | −9 | −12 |
| H2-H1 2 | −15 | −11 | −21 | −15 | −4 | 0 | −8 | −12 | −21 | −22 | −18 | −12 | −12 | −13 |
| HRF 1 | −13 | −8 | −24 | −10 | −9 | −5 | −9 | −12 | −21 | −16 | −14 | −18 | −8 | −11 |
| HRF 2 | −13 | −9 | −21 | −15 | −4 | 0 | −7 | −10 | −20 | −21 | −17 | −11 | −11 | −11 |

The absolute error and its percent with respect to the mean values from the CPIF signal are shown in Table VI. For the non-harmonic measures, the error and its variations were considered sufficiently low (mean error 10%±7%) to make this scheme clinically useful. Particular emphasis is given to the ACC-based AC flow and MFDR estimates, which are indicative measures of vocal hyperfunction when significant variations are noted (for example, by increments larger than 50%). The IBIF accuracy and robustness observed for these two ACC-based estimates is considered adequate to perform such discrimination.

TABLE VI
ESTIMATION ERROR BETWEEN ACC MEASURES AND
THOSE FROM CPIF AND MEASURED VALUES

| | Error-absolute | Error-relative to mean |
|---|---|---|
| Measures | Mean ± Stdv | Mean ± Stdv |
| AC flow | 18.1 ± 19.4 | 7.4% ± 6.5% |
| MFDR | 23.9 ± 18.3 | 9.5% ± 6.6% |
| H2-H1 | 3.3 ± 2.4 | 29.6% ± 27.2% |
| HRF | 3.0 ± 2.1 | 29.3% ± 28.1% |

In light of the evaluation results described above, the subglottal IBIF module provides a concise, yet accurate, method to estimate the glottal airflow and aerodynamic parameters. The modeling mechanism is not encumbered by unnecessary parameters and, thereby, can be readily utilized to evaluate performance parameters, including user-specific calibration, in a manner that is highly effective and efficient.

The scheme yields comparable estimates with respect to the current criterion standard used in clinical settings, particularly for non-harmonic measures. Two measures of interest, MFDR and AC flow, can be accurately estimated using the subglottal IBIF model, and as a result, the subglottal IBIF model is capable of being used to detect vocal hyperfunction. This approach could surpass standard clinical evaluation since it adds the capability to better characterize actual vocal function when individuals engage in their typical daily activities. The subglottal IBIF module could be used directly for the ambulatory monitoring of vocal function. Furthermore, no current ambulatory assessment technique is known to detect vocal hyperfunction. As the scheme is also suitable for real-time biofeedback within this framework, it has the potential as an important tool to improve clinical assessment and treatment of commonly-occurring voice disorders.

The transmission line model of the subglottal system of the present invention, the inclusion of the skin parameters, and the calibration with the oral airflow via waveform matching and RMSE minimization provide improved estimates in comparison to current models. Further implementations of the invention can incorporate changes of skin properties due to neck movements, certain vowel dependency, and other related factors, particularly when applying the method for running speech. For example, the factors that control the changes in the skin properties can be analyzed and used to optimize single values for the ambulatory assessment of vocal function.

In addition, the subglottal IBIF module of the present invention can be incorporated into other applications such as ambulatory vocal biofeedback, speech enhancement, speaker normalization for automatic speech recognition, and/or speaker identification in noise.

The present invention has been described in terms of one or more preferred embodiments, and it should be appreciated that many equivalents, alternatives, variations, and modifications, aside from those expressly stated, are possible and within the scope of the invention.

1. A method for evaluating vocal function of a subject, the method comprising the steps of:
(a) collecting surface acceleration data from an accelerometer coupled to a neck of the subject;
(b) obtaining at least one other physiological indication signal from the subject;
(c) applying an inverse filter to the surface acceleration data based on a basis transmission line model to obtain an estimated glottal airflow waveform;
(d) comparing at least one portion of the estimated glottal airflow waveform to the at least one other physiological signal;
(e) adjusting at least one parameter of the basis transmission line model based on the comparing step to yield a calibrated transmission line model;
(f) reapplying the inverse filter to the surface acceleration data based on the calibrated transmission line model to obtain a new estimated glottal airflow waveform;
(g) repeating at least steps (a) through (c) and analyzing at least one portion of the new estimated glottal airflow waveform against at least a portion of the estimated glottal airflow waveform; and
(h) generating an indication of vocal function of the subject based on at least the analyzing of step (g).

2. The method of claim 1 wherein the basis transmission line model and the calibrated transmission line model are physiological transmission line models representing acoustic impedances of components of the subglottal tract, mechanical impedance of the skin, and radiation impedance due to accelerometer loading, and wherein the transmission line model is decomposed into separate subsections above and below the location of the accelerometer.

3. The method of claim 1 wherein the at least one portion of the estimated glottal airflow waveform includes an estimated first resonance frequency and the at least one other physiological signal includes a calculated first resonance frequency obtained from the surface acceleration data.

4. The method of claim 1 wherein the at least one other physiological signal includes an oral airflow waveform.

5. The method of claim 4 wherein the comparing step includes aligning the at least one portion of the estimated glottal airflow waveform with the oral airflow waveform and calculating a root mean squared error.

6. The method of claim 5 wherein the adjusting step includes adjusting the at least one parameter of the basis transmission line model based to reduce the root mean squared error.

7. The method of claim 1 wherein the at least one parameter includes at least one of air inertance, air viscous resistance, heat conduction resistance, air compliance, soft tissue resistance, soft tissue inertance, soft tissue compliance, cartilage resistance, cartilage inertance, cartilage compliance, skin stiffness, skin mass, and skin resistance.

8. The method of claim 7 wherein the step of adjusting the at least one parameter includes modifying a trachea length measurement.

9. The method of claim 1 and further comprising the step of detecting vocal hyperfunction based on the generated indication of vocal function.

10. The method of claim 1 wherein the at least one portion of the new estimated glottal airflow waveform includes one of an amplitude of unsteady airflow and a maximum flow declination rate.

11. A system to assess vocal function of a subject, the system comprising:
an accelerometer configured to acquire surface acceleration data associated with vocal functionality of the subject; and
a computer system configured to analyze the surface acceleration data and to estimate glottal airflow waveforms produced by the subject based on the surface acceleration data by:
applying an inverse filter to the surface acceleration data based on a basis transmission line model to obtain a first glottal waveform output,
comparing at least one portion of the first glottal waveform output to at least one other physiological signal of the subject,
adjusting at least one parameter in the basis transmission line model based on the comparison step to obtain a calibrated transmission line model,
reapplying the inverse filter to the neck surface acceleration data based on the calibrated transmission line model to obtain the estimated glottal airflow waveforms, and
generating an indication of vocal functionality of the subject based on the estimated glottal airflow waveforms.

12. The system of claim 11 and further comprising a circumferentially vented mask configured to acquire an output airflow waveforms of the subject, and wherein the output airflow waveforms serve as the at least one other physiological signal.

13. The system of claim 12 wherein the comparing includes aligning the at least one portion of the first glottal airflow waveform with the oral airflow waveform and calculating a root mean squared error.

14. The system of claim 11 wherein the at least one other physiological signal is a first resonance frequency derived from the surface acceleration data.

15. The system of claim 11 wherein the basis transmission line model and the calibrated transmission line model are physiological transmission line models representing acoustic impedances of components of a subglottal tract of the subject, mechanical impedance of a skin of the subject, and radiation impedance due to accelerometer loading, and wherein the transmission line model is decomposed into separate subsections based on the location of the accelerometer.

16. The system of claim 11 wherein the indication of vocal functionality of the subject includes an indication of an amplitude of unsteady airflow and a maximum flow declination rate in the estimated glottal airflow waveforms.

17. The system of claim 11 wherein the indication of vocal functionality includes an indication of vocal hyperfunction.

18. The system of claim 11 wherein the adjusting of at least one parameter includes modifying a trachea length measurement.

19. The system of claim 11 wherein the at least one parameter includes at least one of air inertance, air viscous resistance, heat conduction resistance, air compliance, soft tissue resistance, soft tissue inertance, soft tissue compliance, cartilage resistance, cartilage inertance, cartilage compliance, skin stiffness, skin mass, and skin resistance.

20. The system of claim 11 wherein surface acceleration data associated with vocal functionality of the subject includes surface acceleration data from a skin location overlying the subject's suprasternal notch.

21. The system of claim 11 wherein the computer system is configured to perform the comparing, adjusting, and reapplying to perform a subject calibration of the system and repeat the applying and the generating after performing the subject calibration without repeating the comparing, adjusting, and reapplying.
